(19) United States
(12) Patent Application Publication
(10) Pub. No.: US 2008/0064602 A1
(43) Pub. Date: Mar. 13, 2008

(54) Pharmaceutical Composition

(76) Inventors: Ernest Schay, Edenvale (ZA); Walter Focke, Pretoria (ZA); Lushane Walbrugh, Germiston (ZA)

Correspondence Address: SHOOK, HARDY & BACON LLP;INTELLECTUAL PROPERTY DEPARTMENT, 2555 GRAND BLVD, KANSAS CITY, MO, 64108-2613, US

(21) Appl. No.: 11/574,953

(22) PCT Filed: Aug. 27, 2005
(86) PCT No.: PCT/EP05/09264

§ 371 Date: Aug. 27, 2007

(30) Foreign Application Priority Data
Sep. 9, 2004 (ZA) 2004/7208

(51) Int. Cl. A01N 37/32; A01N 25/10; A01P 13/00; A01P 7/02; A61K 31/155; A61K 47/32; A61P 33/14

(52) U.S. Cl. 504/343; 514/631; 514/772.6

(57) ABSTRACT

A pharmaceutical composition comprising an active pharmaceutical composition contained in a water-soluble carrier which is selected from the group comprising polyacrilamide/polyacrylate co-polymer, polyacrylic acid, polyacrylate and mixtures of these is provided.

### FIELD OF THE INVENTION

This invention relates to a pharmaceutical composition and, more particularly, to a pharmaceutical composition in a water-soluble form.

### BACKGROUND TO THE INVENTION

In this specification the term “pharmaceutical” shall have its widest meaning and include compounds used in the treatment of humans, animals and plants. This term includes pesticides and in this specification the term “pesticide” is intended to encompass within its scope herbicides and compositions for eradicating or controlling animal pests.

Liquids in general, and pesticides in particular, are usually sold as a liquid concentrate in metal or plastic containers from which a desired quantity of the product is decanted before use.

Pesticides, by their nature, are hazardous chemicals and disposal of empty pesticide containers is a problem as the containers usually contain a pesticide residue which can leach into the environment with disastrous consequences. To an extent, similar problems can be experienced with full or partially full containers, particularly metal containers or plastic containers that have stripped cap screws. Also, containers frequently fall and break during transport.

Another problem with the containers is the use, particularly in developing countries where amenities such as running water are lacking, of empty containers to store water for domestic use. The dangers of such a practise need no elaboration.

One attempt to address the above problem involves supplying the active pesticide ingredient in powder form in plastic bags. A desired quantity of the active is measured out and mixed with water shortly before using. To a large extent the plastic bags, while requiring less storage space than metal or plastic containers, suffer from the same disadvantages as the containers particularly when they are stored in damp environments and also when measuring the correct quantity of the powder.

### OBJECT OF THE INVENTION

It is an object of this invention to provide a pharmaceutical composition which at least partly alleviates the abovementioned disadvantages.

### SUMMARY OF THE INVENTION

In accordance with this invention there is provided a pharmaceutical composition comprising an active pharmaceutical composition contained in a water-soluble carrier which is selected from the group comprising polyacrilamide/polyacrylate co-polymer, polyacrylic acid, polyacrylate and mixtures of these.

There is further provided for the polyacrylate to be a sodium cross-linked polyacrylate; for the polyacrylate to be present in the range 10 to 20% m/m; for the polyacrilamide/polyacrylate co-polymer to be present in the range 10 to 20% m/m; for the polyacrylic acid to be present in the range 2 to 40% m/m; and for the active pharmaceutical composition to be a pesticide. There is also provided for the pesticide to be a herbicide. Alternatively there is provided for the pesticide to be suitable for controlling or eradicating mammalian pests, preferably ectoparasites and further preferably an acaricide.

There is also provided for the acaricide to be a pesticide belonging to the amidine group of pesticides, preferably AMITRAZ.

There is further provided for the pharmaceutical composition to include at least one suitable dispersant, and/or at least one disintegrants, and/or at least one surfactant and/or at least one filler; for the dispersant to be a sodium salt of the condensation product of naphalene sulphuric acid, preferably WETTOL D2; for the disintegrant to be sodium starch glycolate; and for the surfactant to be a non-ionic surfactant belonging to the nonylphenol ethoxylate group, preferably ARKOPAL N090.

Still further according to this aspect of the invention there is provided for the composition to include AMITRAZ, calcium carbonate, ARKOPAL N090, WETTOL D2, polyacrilamide/polyacrylate co-polymer, preferably HS-800, polyacrylic acid, preferably ASP 4, polyacrylate, preferably Favor Pac, sodium starch glycolate and sodium carbonate; and for the constituents to be present in the following ranges (%m/m):

| | |
|---|---|
| AMITRAZ | 30 to 65% m/m, |
| Calcium carbonate | 8 to 11% m/m, |
| ARKOPAL N090 | 2 to 4% m/m, |
| WETTOL D2 | 1 to 2% m/m, |
| polyacrilamide/polyacrylate co-polymer | 15 to 50% m/m, |
| polyacrylic acid | 2 to 30% m/m, |
| polyacrylate | 10 to 20% m/m, |
| Sodium starch glycolate | 0 to 15% m/m, and |
| Na₂CO₃ | 0.1 to 0.5% m/m. |

### BRIEF DESCRIPTION OF ONE EMBODIMENT OF THE INVENTION

One embodiment of the invention will be described below by way of example only and with reference to the accompanying example which is for an acciricidal composition.

### DETAILED DESCRIPTION OF ONE EMBODIMENT OF THE INVENTION

A pharmaceutical composition according to the invention is formed by mixing a water soluble carrier, an acaricide milled together with calcium carbonate and desired dispersants, surfactants and disintegrants together. Water was added to form a composition having a gel or paste-like constituency and the resultant composition extruded into a desired shape. Each unit so produced is individually sealed with a plastics or foil material film.

The water soluble carriers used in these examples were HS-800, a polyacrilamide/polyacrylate co-polymer, ASP 4, a polyacrylic acid, and Favor Pac, a cross-linked sodium polyacrylate. The acaricide used was AMITRAZ, the surfactants Arkopal NO90 and Wettol D2, and the disintegrant sodium starch glycolate. The constituents were mixed in the following ratios:

| | FORMULATION | | |
|---|---|---|---|
| | 1 | 2 | 3 |
| CONSTITUENT | % m/m | | |
| AMITRAZ | 30 | 51.5 | 55.8 |
| CaCO₃ | 8 | 13.7 | 14.9 |
| ARKOPAL N090 | 2 | 3.4 | 3.3 |
| WETTOL D2 | 1 | 1.75 | 1.9 |
| HS-800 | 47 | 23.3 | — |
| ASP 4 | — | 5.73 | 6.4 |
| Favor Pac | — | — | 16.7 |
| Sodium starch glycolate | 12 | 0.64 | — |
| Na₂CO₃ | — | 0.64 | 0.7 |

Between 10 and 15 ml of water was added to the above dry constituents to provide a gel or paste of suitable consistency.

The concentration of AMITRAZ was based on an effective concentration to control or eradicate acarinids when the dosage unit produced was dissolved in 10 litres of water.

Where ASP 4 (polyacrylic acid) is used in formulation 2, 10 grams of an aqueous solution is added to 5 grams of dry powder constituents. The aqueous solution is formed by neutralising ASP 4 with sodium carbonate and dissolving this in tap water to form a clear viscous fluid. The % m/m (ASP 4+Na₂CO₃) to water is 3.4:96.6.

It is envisaged that the dosage unit can be used as a viable alternative to liquid and powder concentrates thus, at least partly, alleviating some of the disadvantages associated with the use of the liquid and powder. It is envisaged that a number of dosage units can be added to any suitable volume of water to make up enough pesticide for application to one animal, in which case one dosage unit of the pesticide can be made up in a bucket and sprayed or sponged onto the animal, or to several animals, in which case several dosage units of the pesticide can be dissolved in a dip tank or in a spray race reservoir. Alternatively, instead of utilising multiple dosage units in a large dip, a larger (approximately 2-5 kg) amount of dosage form may be packed into a single container.

The consistency of the final product is gel or paste-like thus making it easy to extrude into any suitable shape. This is advantageous in that the size of the dosage unit can be easily changed with only small adjustments to the equipment used. This is unlike conventional tabletting procedures where a change in tablet size requires expensive changes to dies and presses. Also, suppository technology can be used to form the dosage unit.

Also, any suitable container or packaging can be used. It is, for example, envisaged that cartons, “doy” or stand alone bags, and blister packs can be used.

It will be appreciated that a wide variety of active pharmaceutical ingredients and additives can be used in the above invention without departing from the scope thereof. In particular, the active ingredient can be an acaricide as described or the pesticide may target other ectoparasites. Alternatively the active ingredient may target a variety of endoparasites such as nematodes and cystodes to name but two. Where the target is an endoparasite it is envisaged that the tablet can be dissolved in water in a reservoir for an oral dosing gun.

In addition to the above, the active ingredient may also be a herbicide and the dosage unit dissolved in water in a suitable reservoir shortly before it is applied. In a similar fashion, the active ingredient may be a protein, disinfectant or any other pharmacologically active material.

1. A pharmaceutical composition comprising an active pharmaceutical composition contained in a water-soluble carrier which is selected from the group consisting of polyacrilamide/polyacrylate co-polymer, polyacrylic acid, polyacrylate and mixtures of these.

2. A pharmaceutical composition as claimed in claim 1 in which the polyacrylate is a sodium cross-linked polyacrylate.

3. A pharmaceutical composition as claimed in claim 1 or claim 2 in which the polyacrylate is present in the range 10 to 20% m/m.

4. A pharmaceutical composition as claimed in any one of the preceding claims in which the polyacrilamide/polyacrylate co-polymer is present in the range 15 to 50% m/m.

5. A pharmaceutical composition as claimed in any one of the preceding claims in which the polyacrylic acid is present in the range 2 to 20% m/m.

6. A pharmaceutical composition as claimed in any one of the preceding claims in which the active pharmaceutical composition is a pesticide.

7. A pharmaceutical composition as claimed in claim 6 in which the pesticide is a herbicide.

8. A pharmaceutical composition as claimed in claim 6 in which the pesticide is suitable for controlling or eradicating mammalian pests.

9. A pharmaceutical composition as claimed in claim 8 in which the pesticide is suitable for controlling or eradicating ectoparasites.

10. A pharmaceutical composition as claimed in claim 8 in which the pesticide is an acaricide.

11. A pharmaceutical composition as claimed in claim 10 in which the acaricide belongs to the amidine group of pesticides.

12. A pharmaceutical composition as claimed in claim 11 in which the pesticide is AMITRAZ.

13. A pharmaceutical composition as claimed in claim 12 in which the AMITRAZ is present in the range 30 to 65% m/m.

14. A pharmaceutical composition as claimed in any one of the preceding claims which further includes any one or more of at least one dispersant, at least one disintegrant, at least one surfactant and at least one filler.

15. A pharmaceutical composition as claimed in claim 14 in which the dispersant is a sodium salt of the condensation product of naphalene sulphuric acid.

16. A pharmaceutical composition as claimed in claim 15 in which the dispersant is WETTOL D2.

17. A pharmaceutical composition as claimed in any one of claims 14 to 16 in which the dispersant is present in the range 1 to 2% m/m.

18. A pharmaceutical composition as claimed in any one of claims 14 to 17 in which the surfactant is a non-ionic surfactant belonging to the nonylphenol ethoxylate group.

19. A pharmaceutical composition as claimed in claim 18 in which the surfactant is ARKOPAL N090.

20. A pharmaceutical composition as claimed in any one of claims 14 to 19 in which the surfactant is present in the range 2 to 4% m/m.

21. A pharmaceutical composition as claimed in any one of claims 14 to 20 in which the disintegrant is sodium starch glycolate.

22. A pharmaceutical composition as claimed in claim 21 in which the sodium starch glycolate is present in the range 0 to 15% m/m.

23. A pharmaceutical composition as claimed in any one of claims 14 to claim 22 which includes calcium carbonate present in the range 5 to 20% m/m.
